SEQUENCE LISTING (40 sequences)

SEQ ID NO: 1 - DNA, 26 bp, Artificial
  MS-PCR primer
ggttagtagttcgggtagggtttatc

SEQ ID NO: 2 - DNA, 18 bp, Artificial
  MS-PCR primer
ccgaatcctaccccgacg

SEQ ID NO: 3 - DNA, 24 bp, Artificial
  MS-PCR primer
ttagtagtttgggtagggtttatt

SEQ ID NO: 4 - DNA, 18 bp, Artificial
  MS-PCR primer
ccaaatcctaccccaaca

SEQ ID NO: 5 - DNA, 20 bp, Artificial
  MS-PCR primer
gtcgagaacgcgagcgattc

SEQ ID NO: 6 - DNA, 20 bp, Artificial
  MS-PCR primer
cgaccaatccaaccgaaacg

SEQ ID NO: 7 - DNA, 23 bp, Artificial
  MS-PCR primer
gatgttgagaatgtgagtgattt

SEQ ID NO: 8 - DNA, 19 bp, Artificial
  MS-PCR primer
aaccaatccaaccaaaaca

SEQ ID NO: 9 - DNA, 27 bp, Artificial
  MS-PCR primer
gtgtatttggatagtagtaagttcgtc

SEQ ID NO: 10 - DNA, 23 bp, Artificial
  MS-PCR primer
cgtaaaaaaaaccgatctaaccg

SEQ ID NO: 11 - DNA, 27 bp, Artificial
  MS-PCR primer
ggtgtatttggatagtagtaagtttgt

SEQ ID NO: 12 - DNA, 24 bp, Artificial
  MS-PCR primer
ccataaaaaaaaccaatctaacca

SEQ ID NO: 13 - DNA, 24 bp, Artificial
  MS-PCR primer
ttattagagggtggggcggatcgc

SEQ ID NO: 14 - DNA, 21 bp, Artificial
  MS-PCR primer
gaccccgaaccgcgaccgtaa

SEQ ID NO: 15 - DNA, 24 bp, Artificial
  MS-PCR primer
ttattagagggtggggtggattgt

SEQ ID NO: 16 - DNA, 22 bp, Artificial
  MS-PCR primer
caaccccaaaccacaaccataa

SEQ ID NO: 17 - DNA, 25 bp, Artificial
  MS-PCR primer
ggttaatttagagtttcgagagacg

SEQ ID NO: 18 - DNA, 24 bp, Artificial
  MS-PCR primer
tcaacgaactcacgccgcgcaatc

SEQ ID NO: 19 - DNA, 25 bp, Artificial
  MS-PCR primer
ggttaatttagagttttgagagatg

SEQ ID NO: 20 - DNA, 25 bp, Artificial
  MS-PCR primer
tcaacaaactcacaccacacaatca

SEQ ID NO: 21 - DNA, 19 bp, Artificial
  MS-PCR primer
acgggttatttttttttcg

SEQ ID NO: 22 - DNA, 20 bp, Artificial
  MS-PCR primer
taaaatatacgccctccacg

SEQ ID NO: 23 - DNA, 19 bp, Artificial
  MS-PCR primer
atgggttattttttttttg

SEQ ID NO: 24 - DNA, 20 bp, Artificial
  MS-PCR primer
taaaatatacaccctccaca

SEQ ID NO: 25 - DNA, 20 bp, Artificial
  MS-PCR primer
tgattgtcgttcgtagtacg

SEQ ID NO: 26 - DNA, 20 bp, Artificial
  MS-PCR primer
cgacaatttaataacacgcg

SEQ ID NO: 27 - DNA, 20 bp, Artificial
  MS-PCR primer
tgattgttgtttgtagtatg

SEQ ID NO: 28 - DNA, 20 bp, Artificial
  MS-PCR primer
caacaatttaataacacaca

SEQ ID NO: 29 - DNA, 18 bp, Artificial
  MS-PCR primer
gggttttgcgagagcgcg

SEQ ID NO: 30 - DNA, 18 bp, Artificial
  MS-PCR primer
gctaacaaacgcgaaccg

SEQ ID NO: 31 - DNA, 22 bp, Artificial
  MS-PCR primer
ggttttgtgagagtgtgtttag

SEQ ID NO: 32 - DNA, 22 bp, Artificial
  MS-PCR primer
cactaacaaacacaaaccaaac

SEQ ID NO: 33 - DNA, 21 bp, Artificial
  MS-PCR primer
gtttagttttgaggggggcgc

SEQ ID NO: 34 - DNA, 21 bp, Artificial
  MS-PCR primer
aacttcctactacgaccgacg

SEQ ID NO: 35 - DNA, 23 bp, Artificial
  MS-PCR primer
attgtaaagtgaaggtgtgggtt

SEQ ID NO: 36 - DNA, 23 bp, Artificial
  MS-PCR primer
ccaacttcctactacaaccaaca

SEQ ID NO: 37 - DNA, 25 bp, Artificial
  MS-PCR primer
ttgggaatttagttgtcgtcgtttc

SEQ ID NO: 38 - DNA, 25 bp, Artificial
  MS-PCR primer
aaacaacgactaccgatactacgcg

SEQ ID NO: 39 - DNA, 28 bp, Artificial
  MS-PCR primer
gtgttgggaatttagttgttgttgtttt

SEQ ID NO: 40 - DNA, 30 bp, Artificial
  MS-PCR primer
actaccaaaacaacaactaccaatactaca

(19) United States
(12) Patent Application Publication
(10) Pub. No.: US 2010/0124742 A1
(43) Pub. Date: May 20, 2010

(54) Methods and Compositions to Identify Increased Risk of Breast Cancer by Detection of CPG Island Methylator Phenotype (CIMP)

(75) Inventor: Victoria L. Seewaldt, Durham, NC (US)

Correspondence Address: MYERS BIGEL SIBLEY & SAJOVEC, PO BOX 37428, RALEIGH, NC, 27627, US

(73) Assignee: DUKE UNIVERSITY

(21) Appl. No.: 12/512,296

(22) Filed: Jul. 30, 2009

Related U.S. Application Data
(60) Provisional application No. 61/085,155, filed on Jul. 31, 2008.

(51) Int. Cl. C12Q 1/68

(52) U.S. Cl. 435/6

(57) ABSTRACT

The present invention is directed to kits and methods for identifying a female subject as having an increased risk of developing breast cancer, comprising detecting a CPG island methylator phenotype (CIMP) in nucleic acid of the subject.

### STATEMENT OF PRIORITY

This application claims the benefit, under 35 U.S.C. §119(e), of U.S. Provisional Application Ser. No. 61/085,155, filed Jul. 31, 2008, the entire contents of which are incorporated by reference herein.

### STATEMENT OF GOVERNMENT SUPPORT

Aspects of the present invention were made with the support of federal finding under grant numbers CA68438-AV13 (AVON/NCI Partners in Progress), 2P30CA14236-26, R01CA88799, R01CA98441, and R01CA114068 from the National Institutes of Health/National Cancer Institute (NIH/NCI). The United States Government has certain rights in this invention.

### FIELD OF THE INVENTION

The present invention relates to methods and compositions directed to the identification of subjects at increased risk of breast cancer.

### BACKGROUND OF THE INVENTION

Transcriptional silencing of tumor suppressor genes (TSGs) through methylation of CpG islands in promoter regions is thought to be an important early mechanism of human carcinogenesis (1, 2). Growing evidence suggests that epigenetic inactivation via cytosine methylation plays a role in the transformation of normal cells to cancerous cells, underscoring the need to investigate global CpG island methylation patterns. Building on these observations, Toyota et al. proposed the existence of a CpG Island Methylator Phenotype (CIMP), which leads to cancer development through the simultaneous inactivation of multiple tumor suppressor genes and induction of mismatch repair deficiency (3). In studies of colorectal cancer, an established panel of CIMP-specific promoters (MINT1 [Methylated in Tumors-1], MINT2 [Methylated in Tumors-2], MINT31 [Methylated in Tumors-31], INK4a/ARF, and hMLH1) has been used to distinguish between a CIMP-low (zero or one markers methylated) and CIMP-high phenotype (2 or more of 5 markers methylated) (4-7). Studies by Weisenberger et al. defined the CIMP phenotype in colon cancer, testing 200 methylation markers in 295 colon cancer specimens (8).

There remains a need to identify subjects at increased risk of breast cancer. Furthermore, biomarkers are needed in order to accurately predict short-term breast cancer risk so that women who are most likely to benefit from preventative therapy can be identified, and so that response to chemoprevention can be accurately assessed. Accordingly, the present invention provides methods and compositions for identifying subjects at increased risk of breast cancer by detection of a CPG island methylator phenotype (CIMP).

### SUMMARY OF THE INVENTION

Accordingly, a first aspect of the invention is a method of identifying a female subject as having an increased risk of developing breast cancer, comprising detecting methylation of one or more CpG islands in the promoter of a HIN-1 gene, in the M4 region of the promoter element of a RARB gene and in the promoter of an INK4a/ARF gene in the subject, whereby the presence of methylation of one or more CpG islands in the promoter of the HIN-1 gene, in the M4 region of the promoter element of the RARB gene and in the promoter of the INK4a/ARF gene identifies the subject as having an increased risk of developing breast cancer.

A second aspect of the invention provides a method of identifying a female subject as having an increased risk of developing breast cancer, comprising detecting methylation of one or more CpG islands in the promoter of a HIN-1 gene, in the M4 region of the promoter element of a RARB gene, in the promoter of an INK4a/ARF gene and in the promoter of a progesterone receptor alpha (PRA) gene in the subject, whereby the presence of methylation of one or more CpG islands in the promoter of the HIN-1 gene, in the M4 region of the promoter element of the RARB gene, in the promoter of the INK4a/ARF and in the promoter of the PRA gene identifies the subject as having an increased risk of developing breast cancer.

A further aspect of the invention is a kit for the detection of methylation of one or more CpG islands in the promoter of a HIN-1 gene, in the M4 region of the promoter element of a RARB gene and in the promoter of an INK4a/ARF gene, comprising reagents and primers for amplification of CpG-containing nucleic acid of the HIN-1 gene, of CpG-containing nucleic acid of the RARB M4 gene and of CpG-containing nucleic acid of the INK4a/ARF gene. In some embodiments, the kit of this invention comprises reagents and primers for amplification of CpG-containing nucleic acid of the PRA gene.

The present invention will now be described more fully hereinafter with reference to the accompanying drawings and specification, in which various embodiments of the invention are shown. This invention may, however, be embodied in different forms and should not be construed as limited to the embodiments set forth herein.

### BRIEF DESCRIPTION OF THE FIGURES

FIGS. 1A-C show the frequency and distribution of methylation events in high-risk women. A total of 109 women were tested for methylation of 10 promoter/promoter element targets: RARB (M3 and M4), ESR1, INK4a/ARF, BRCA1, PRA, PRB, RASSF1A, HIN-1, and CRBP1. (A) shows representative methylation in Random Periareolar Fine Needle Aspiration (RPFNA) cytology. Methylation of the ESR1 promoter in RPFNA obtained from 15 representative high-risk women with non-proliferative, hyperplastic, or atypical RPFNA. M and U denote the use of methylation specific-polymerase chain reaction (MS-PCR) primers to identify methylated and unmethylated ESR1 promoter, respectively. (+) denotes a methylated positive control in the M gels and the T47D breast cancer cell line in the U gels. (−) denotes the negative control. (B) shows methylation events in women with non-proliferative (Masood score≦10), hyperplastic (Masood score 11-13), and atypical Masood cytology (Masood score≧14). A dark gray bar denotes bilateral methylation, a light gray bar denotes unilateral methylation, and a white bar denotes no promoter/promoter element methylation detected. These data are the summary of three independent tests. np=non-proliferative. (C) shows the frequency of promoter/promoter element methylation in high-risk women.

FIGS. 2A-B show patterns of correlation among ten promoter/promoter element methylation markers from RPFNA cytology. The association between all pair-wise combinations of ten markers was examined in 189 RPFNA samples that had no missing data on promoter/promoter element methylation. The ten promoter/promoter element targets are RARB (M3 and M4), ESR1, INK4a/ARF, BRCA1, PRA, PRB, RASSF1A, HIN-1, and CRBP1. (A) shows a dendrogram for the agglomerative hierarchical clustering of promoter/promoter element methylation states, showing patterns of similarity observed among markers. (B) shows the agreement evaluation by the Fishers Exact Test for all 45 pairs of markers. Results are provided as estimated odds ratios and adjusted p-values, after correcting for the (False Discovery Rate) FDR at the alpha=0.05 level. Significant pair-wise correlations are highlighted in grey.

FIGS. 3A-B show that the frequency of promoter/promoter element methylation is inversely correlated with likelihood of carrying a BRCA1/2 mutation. (A) shows the distribution and frequency of methylation events in 25 women testing negative (black) and 15 women testing positive (grey) for a BRCA1/2 mutation. (B) shows the number and percentage of women methylated for each marker out of the 15 women testing positive for BRCA1/2.

### DETAILED DESCRIPTION

The present invention is based on the discovery of a specific CIMP associated with breast cancer initiation and non-BRCA1/2 familial breast cancer. CIMP was tested for in early mammary carcinogenesis by analyzing a panel of ten methylation markers in mammary epithelial cells from 109 asymptomatic women at increased risk for breast cancer. The frequency of promoter/promoter element methylation was tested in Random Periareolar Fine Needle Aspiration (RPFNA) cytology and classified relative to Masood Cytology Index (MCI) and age. RPFNA is a research technique developed to repeatedly sample mammary cells from the whole breast of asymptomatic women at high risk for development of breast cancer, so as to assess both 1) breast cancer risk and 2) response to chemoprevention (11-13). RPFNA can be performed successfully in a majority of high-risk women (82-89% cell yield). RPFNA samples were classified using the Masood Cytology Index to indicate the level of cytological abnormality.

Thus, the present invention is based on the discovery of a correlation between methylation (e.g., CpG methylation) of (1) the M4 region of the promoter element of the retinoic acid receptor-beta2 (RARB) gene, (2) the promoter of the inhibitor of cdk4/p16/alternate reading frame (INK4a/ARF) gene, and (3) the promoter of the high in normal-1 (HIN-1) gene in a female subject and an increased risk of developing breast cancer. Thus, in one aspect, the present invention provides a method of identifying a female subject as having an increased risk of developing breast cancer, comprising detecting methylation of one or more CpG islands in the promoter of a HIN-1 gene, in the M4 region of the promoter element of a RARB gene and in the promoter of an INK4a/ARF gene in the subject, whereby the presence of methylation of one or more CpG islands in the promoter of the HIN-1 gene, in the M4 region of the promoter element of the RARB gene and in the promoter of the INK4a/ARF gene identifies the subject as having an increased risk of developing breast cancer.

In some embodiments, the present invention provides a method of identifying a female subject as having an increased risk of developing breast cancer, comprising detecting methylation of one or more CpG islands in the promoter of a HIN-1 gene, in the M4 region of the promoter element of a RARB gene and/or in the promoter of an INK4a/ARF gene in the subject, in any combination, whereby the presence of methylation of one or more CpG islands in the promoter of the HIN-1 gene, in the M4 region of the promoter element of the RARB gene and/or in the promoter of the INK4a/ARF gene in any combination identifies the subject as having an increased risk of developing breast cancer.

A further aspect of the invention provides a method of identifying a female subject as having an increased risk of developing breast cancer, comprising detecting methylation of one or more CpG islands in the promoter of a HIN-1 gene, in the M4 region of the promoter element of a RARB gene, in the promoter of an INK4a/ARF gene and in the promoter of a progesterone receptor alpha (PRA) gene in the subject, whereby the presence of methylation of one or more CpG islands in the promoter of the HIN-1 gene, in the M4 region of the promoter element of the RARB gene, in the promoter of the INK4a/ARF and in the promoter of the PRA gene identifies the subject as having an increased risk of developing breast cancer.

The present invention further provides a method of identifying a female subject as having an increased risk of developing breast cancer, comprising detecting methylation of one or more CpG islands in the promoter of a HIN-1 gene, in the M4 region of the promoter element of a RARB gene, in the promoter of an INK4a/ARF gene and/or in the promoter of a progesterone receptor alpha (PRA) gene in the subject, in any combination, whereby the presence of methylation of one or more CpG islands in the promoter of the HIN-1 gene, in the M4 region of the promoter element of the RARB gene, in the promoter of the INK4a/ARF and/or in the promoter of the PRA gene, in any combination, identifies the subject as having an increased risk of developing breast cancer.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount (e.g., an amount of methylation) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim of this invention is not intended to be interpreted to be equivalent to “comprising.”

The term “genetic marker” as used herein refers to a region of a nucleotide sequence (e.g., in a chromosome) that is subject to variability (i.e., the region can be polymorphic for a variety of alleles).

An “allele” as used herein refers to one of two or more alternative forms of a nucleotide sequence at a given position (locus) on a chromosome. Usually alleles are nucleotide sequences that make up the coding sequence of a gene, but sometimes the term is used to refer to a nucleotide sequence in a non-coding sequence. An individual's genotype for a given gene is the set of alleles it happens to possess.

As used herein, “nucleic acids” encompass both RNA and DNA, including cDNA, genomic DNA, mRNA, synthetic (e.g., chemically synthesized) DNA and chimeras of RNA and DNA. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

An “isolated nucleic acid” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter/promoter element) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

An isolated cell refers to a cell that is separated from other cells and/or tissue components with which it is normally associated in its natural state. For example, an isolated cell is a cell that is part of a cell culture. An isolated cell can also be a cell that is administered to or introduced into a subject, e.g., to impart a therapeutic or otherwise beneficial effect.

The term “oligonucleotide” refers to a nucleic acid sequence of at least about six nucleotides to about 100 nucleotides, for example, about 15 to 30 nucleotides, or about 20 to 25 nucleotides, which can be used, for example, as a primer in an amplification reaction (e.g., PCR) and/or as a probe in a hybridization assay and/or in a microarray. Oligonucleotides can be natural or synthetic, e.g., DNA, RNA, modified backbones, etc., as are well known in the art. Peptide nucleic acids (PNAs) can also be used as oligonucleotides (e.g., as probes) in the methods of this invention.

A subject of this invention is any animal that is susceptible to breast cancer as defined herein. Examples of subjects of this invention can include, but are not limited to, humans, mammals, non-human primates, dogs, cats, horses, cows, goats, guinea pigs, mice, rats and rabbits, as well as any other domestic or commercially valuable animal including animal models of breast cancer. Although the subject of this invention can be either gender, in particular embodiments, the subject is a female subject. Thus, in some embodiments, a subject of the invention can be a female human, a female non-human primate, a female dog, a female cat, and the like, as well as any other domestic or commercially valuable animal including female animal models of breast cancer. In certain embodiments, the subject can be a pre-menopausal, peri-menopausal, menopausal or post-menopausal female. In particular embodiments, wherein the subject is a human female, the subject can be of any ethnicity (e.g., Caucasian, African-American, African, European-American, white, black, Hispanic and/or Asian). A subject of the present invention can further be a subject of this invention that has been previously identified as being at high risk of developing breast cancer. Alternatively, a subject of the present invention can be a subject that has not been previously identified as being at high risk of developing breast cancer.

CpG islands are genomic regions that contain a high frequency of CG dinucleotides. Thus, these regions generally have a GC percentage that is greater than about 50% and with an observed/expected CpG ratio that is greater than about 60%. (Gardiner-Garden et al. “CpG islands in vertebrate genomes,” J Mol Biol 196: 261-282 (1987)).

As used herein, the term “methylation” refers to the presence of epigenetic methylation of cytosine residues in DNA at sites where it is not typically present in normal cells.

Also as used herein the terms “promoter” and “promoter element” refer to DNA sequences that regulate the expression of a gene; these sequences can be upstream or downstream or at any location relative to the transcriptional initiation site of the gene, from which they provide their regulatory effect. A nonlimiting example of a promoter element of this invention is in the M4 region of the RARB gene.

Detection of methylation of the CpG islands can be carried out according to methods of this invention, as well as any art-known method, including, but not limited to, e.g., methylation specific-polymerase chain reaction (MS-PCR), a method of nucleic acid amplification that is well known in the art. In this assay, bisulfite modification of the DNA sequence allows the detection of differences between methylated and unmethylated alleles. Reaction of the DNA with sodium bisulfite converts all unmethylated cytosines to uracil, which is recognized as thymine by Taq polymerase, but does not affect methylated cytosines. Amplification with primers specific for methylated or unmethylated DNA discriminates between methylated and unmethylated DNA. This assay provides a simple and fast way of surveying multiple samples to detect methylation of cytosines in the region of interest (Widschwendter et al., “Methylation and silencing of the retinoic acid receptor-beta2 gene in breast cancer” J. Natl. Cancer Inst. 92(10):826-832 (2000)).

Other methods known in the art for detection and/or quantitative analysis of DNA methylation include, but are not limited to, the chromatin immunoprecipitation assay (ChIP) (Mulero-Navarro et al., Carcinogenesis 27:1099-1104 (2006); Nakagawachi et al., Oncogene 22:8835-8844 (2003)); MethyLight®, a bisulfite modification-dependent fluorescence-based real time PCR assay (Eads et al. Nucleic Acids Res. 28(8) e32 (2000); Erhlich et al., Oncogene 21:6694-6702 (2002)); pyrosequencing (Lee et al. Clinical Cancer Research 14:2664-2672 (2008); Dejeux et al., J. Mol. Diagn. 9:510-520 (2007)); and the Sequenom® MassARRAY® system (Sequenom, Inc., San Diego, Calif.), which utilizes MALDI-TOF mass spectrometry in combination with RNA base specific cleavage (MassCLEAVE™ kit) (Sequenom, Inc., San Diego, Calif.).

A subject that has been previously identified as being at high risk for breast cancer is a subject identified as having at least one major risk factor for breast cancer prior to being tested for methylation of one or more CpG islands in the promoter of the HIN-1 gene, in the M4 region of the promoter element of a RARB gene, and in the promoter of the INK4a/ARF and in some embodiments, in the promoter of the PRA gene. The major risk factors for breast cancer include, but are not limited to: (1) 5-year Gail risk calculation ≧1.7%, (2) prior biopsy exhibiting atypical hyperplasia, lobular carcinoma in situ (LCIS), ductal carcinoma in situ (DCIS), and/or (3) known or suspected BRCA1/2 mutation carrier. A subject of this invention can also be at high risk for breast cancer because of a family history of breast cancer.

Likewise, a subject that has not been previously identified as being at high risk for breast cancer is a subject who has not been identified as having at least one major risk factor for breast cancer prior to being tested for methylation of one or more CpG islands in the promoter of the HIN-1 gene, in the M4 region of the promoter element of a RARB gene and in the promoter of the INK4a/ARF gene and in some embodiments, in one or more CpG islands in the promoter of the PRA gene.

In some embodiments of the invention, the subject of this invention can be negative for a BRCA1 (Breast Cancer Gene 1) mutation. In some embodiments of the invention, the subject can be negative for a BRCA2 (Breast Cancer Gene 2) mutation. In further embodiments of the invention, the subject can be negative for both a BRCA1 mutation and a BRCA2 mutation. A subject is identified as being positive for BRCA1, BRCA2 or both BRCA1 and BRCA2 according to methods well known in the art (Malone et al. “BRCA1 mutations and breast cancer in the general population: Analyses in women before age 35 years and in women before age 45 years with first-degree family history” Journal of the American Medical Association 1998; 279(12):922-929; Martin and Weber. “Genetic and hormonal risk factors in breast cancer” Journal of the National Cancer Institute 2000; 92(14):1126-1135; Newman et al. “Frequency of breast cancer attributable to BRCA1 in a population-based series of American women” Journal of the American Medical Association 1998; 279(12):915-921; Peshkin et al. “BRCA1/2 testing: Complex themes in result interpretation” Journal of Clinical Oncology 2001; 19(9):2555-2565).

In various embodiments of this invention, methylation can be detected in either one breast (unilateral methylation) or both breasts (bilateral methylation). Furthermore the number of methylation events can be 1, 2, 3, 4, 5, 6, 7, 8, 9, 10, etc.

A further embodiment of the invention provides a method for identifying tamoxifen resistance in a female subject, comprising detecting methylation of one or more CpG islands in the promoter of a HIN-1 gene, in the M4 region of the promoter element of a RARB gene, in the promoter of an INK4a/ARF gene and in the promoter of a progesterone receptor alpha (PRA) gene in the subject, whereby the presence of methylation of one or more CpG islands in the promoter of the HIN-1 gene, in the M4 region of the promoter element of the RARB gene, in the promoter of the INK4a/ARF gene and in the promoter of the PRA gene identifies tamoxifen resistance in the subject.

Also provided herein is a method for identifying tamoxifen resistance in a female subject, comprising detecting methylation of one or more CpG islands in the promoter of a HIN-1 gene, in the M4 region of the promoter element of a RARB gene, in the promoter of an INK4a/ARF gene and/or in the promoter of a progesterone receptor alpha (PRA) gene in the subject, in any combination, whereby the presence of methylation of one or more CpG islands in the promoter of the HIN-1 gene, in the M4 region of the promoter element of the RARB gene, in the promoter of the INK4a/ARF gene and/or in the promoter of the PRA gene, in any combination, identifies tamoxifen resistance in the subject.

An additional embodiment of the invention provides a method for identifying a female subject having an increased risk of progression to breast cancer, comprising detecting methylation of one or more CpG islands in the promoter of a HIN-1 gene, in the M4 region of the promoter element of a RARB gene, in the promoter of an INK4a/ARF gene and in the promoter of a progesterone receptor alpha (PRA) gene in the subject, whereby the presence of methylation of one or more CpG islands in the promoter of the HIN-1 gene, in the M4 region of the promoter element of the RARB gene, in the promoter of the INK4a/ARF gene and in the promoter of the PRA gene identifies the subject as having an increased risk of progression to breast cancer. A subject of this method can be a subject that has been previously identified as being at high risk of developing breast cancer and/or a subject in which atypia is detected (Fabian et al. “Short-term breast cancer prediction by random periareolar fine-needle aspiration cytology and the Gail risk model” J. Natl. Cancer Inst. 2000 92(15):1217-27).

An additional embodiment of the invention provides a method for identifying a female subject having an increased risk of progression to breast cancer, comprising detecting methylation of one or more CpG islands in the promoter of a HIN-1 gene, in the M4 region of the promoter element of a RARB gene, in the promoter of an INK4a/ARF gene and in the promoter of a progesterone receptor alpha (PRA) gene in the subject, whereby the presence of methylation of one or more CpG islands in the promoter of the HIN-1 gene, in the M4 region of the promoter element of the RARB gene, in the promoter of the INK4a/ARF gene and in the promoter of the PRA gene identifies the subject as having an increased risk of progression to breast cancer. A subject of this method can be a subject that has been previously identified as being at high risk of developing breast cancer and/or a subject in which atypia is detected (Fabian et al. “Short-term breast cancer prediction by random periareolar fine-needle aspiration cytology and the Gail risk model” J. Natl. Cancer Inst. 2000 92(15):1217-27).

Further provided herein is a method for identifying a female subject having an increased risk of progression to breast cancer, comprising detecting methylation of one or more CpG islands in the promoter of a HIN-1 gene, in the M4 region of the promoter element of a RARB gene, in the promoter of an INK4a/ARF gene and/or in the promoter of a progesterone receptor alpha (PRA) gene in the subject, in any combination, whereby the presence of methylation of one or more CpG islands in the promoter of the HIN-1 gene, in the M4 region of the promoter element of the RARB gene, in the promoter of the INK4a/ARF gene and/or in the promoter of the PRA gene, in any combination, identifies the subject as having an increased risk of progression to breast cancer. A subject of this method can be a subject that has been previously identified as being at high risk of developing breast cancer and/or a subject in which atypia is detected (Fabian et al. “Short-term breast cancer prediction by random periareolar fine-needle aspiration cytology and the Gail risk model” J. Natl. Cancer Inst. 2000 92(15):1217-27).

In addition, the present invention provides a kit comprising reagents for the detection of methylation of one or more CpG islands in the promoter of a HIN-1 gene, in the M4 region of the promoter element of a RARB gene and in the promoter of an INK4a/ARF gene. In further embodiments, the kit can also comprise reagents for the detection of methylation of CpG islands in the promoter of a PAR gene.

Accordingly, in certain embodiments, the kit comprise reagents and primers (and optionally probes) for amplification and analysis of CpG-containing nucleic acid of the promoter of the HIN-1 gene, of CpG-containing nucleic acid of the M4 region of the promoter element of the RARB gene, and of CpG-containing nucleic acid of the promoter of the INK4a/ARF gene. In other embodiments, the kit also comprises reagents and primers (and optionally probes) for amplification and analysis of CpG-containing nucleic acid of the promoter of the PRA gene. Examples of primers that can be employed in the methods and kits of the invention include but are not limited to those listed in Example 2, Table 1, below.

The present invention is more particularly described in the Examples set forth below, which are not intended to be limiting of the embodiments of this invention.

### EXAMPLES

### Example 1

### Random Periareolar Fine Needle Aspiration (RPFNA) and Mathematical Assessment of Breast Cancer Risk

To be eligible for Random Periareolar Fine Needle Aspiration (RPFNA), women were required to have at least one of the following major risk factors for breast cancer: (1) 5-year Gail risk calculation ≧1.7%, (2) prior biopsy exhibiting atypical hyperplasia, lobular carcinoma in situ (LCIS), ductal carcinoma in situ (DCIS), or (3) being a known or suspected BRCA1/2 mutation carrier (13).

BRCAPRO score and Gail model assessments were performed using the CancerGene® software and Breast Cancer Risk Assessment Tool (14, 15). The 5-year breast cancer risk calculated by the Gail model identifies women who are at increased risk compared to their age- and race-matched peers (16). Women under age 35 are not appropriate for Gail risk calculation. The Gail risk calculation was not performed for African-American women because of the potential underestimation of risk in this population. The BRCAPRO model calculates the probability of an individual carrying a mutation in the BRCA1 or BRCA2 genes, using Bayesian methods to incorporate relevant family history, including second-degree relatives, of breast and/or ovarian cancers (17).

RPFNA: RPFNA was performed as previously described (11-13). A minimum of one epithelial cell cluster with at least ten epithelial cells was required to sufficiently determine pathology; the most atypical cell cluster was examined and scored (11, 13). Cells were classified qualitatively as non-proliferative, hyperplasia, or hyperplasia with atypia (18). Cytology preparations were also given a semiquantitative index score through evaluation by the Masood Cytology Index (19). As previously described, cells were given a score of 1-4 points for each of six morphological characteristics that include cell arrangement, pleomorphism, number of myoepithelial cells, anisonucleosis, nucleoli, and chromatin clumping; the sum of these points computed the Masood score: ≦10, non-proliferative (normal); 11-13, hyperplasia; 14-17, atypia; >17, suspicious cytology (13, 19). The number of epithelial cells was quantified and classified as <10 cells (insufficient quantity for cytological analysis), 10-100 cells, 100-500 cells, 500-1,000 cells, 1,000-5,000 cells, and >5,000 cells. Morphological assessment, Masood Cytology Index scores, and cell count were assigned by a blinded, single dedicated pathologist (13).

Masood and Methylation Assessment of Individuals Undergoing Unilateral or Bilateral RPFNA: Twenty-five (25/109) women who had prior mastectomy or radiation therapy underwent unilateral RPFNA (contralateral breast only); eighty-four (84/109) women had neither mastectomy nor radiation therapy and underwent bilateral RPFNA. For women with bilateral RPFNA, the highest Masood score and cell count was considered for this analysis.

Materials and Cell Culture Lines: Sodium bisulfite (Sigma, St. Louis, Mo.; A.C.S.) and hydroquinone (Sigma, 99+%) were used under reduced lighting and stored in a desiccator. Positive and negative control breast cancer cell lines were grown in supplemented alphaMEM (Life Technologies, Gaithersburg, Md.) (20).

DNA Extraction and Bisulfite Treatment: DNA was extracted from breast cancer cell lines and RPFNA as previously described; bisulfite treatment was as previously described (12).

### Example 2

### Methylation Specific Polymerase Chain Reaction (MS-PCR)

All PCR reactions consisted of 50 ng bisulfite-treated DNA, 1×PCR buffer, 250 μM of each dNTP, 200 nM of each primer, and 2.5 U of HotStar® Taq polymerase (Qiagen, Valencia, Calif.) in 30 μl total volume. To estimate PCR sensitivity, titrated experiments were performed using known amounts of methylated, genomic positive control DNA (1 μg-100 pg) spiked in unmethylated genomic DNA for a total of 1 μg. Ten MS-PCR promoter methylation targets were tested: RARB at M3 (nt −51 to nt +162) and M4 (nt +104 to nt +251) (GenBank® Accession No. X56849) (12), BRCA1 (nt −150 to nt +32) (GenBank® Accession No. L78833) (21), ESR1 (nt +367 to nt +494) (GenBank® Accession No. X62462)(22), INK4a/ARF (nt +171 to nt +312) (GenBank® Accession No. X94154) (23, 24), PRA (nt +910 to nt +1008) (GenBank® Accession No. AY525610) (25, 26), PRB (nt +156 to nt +355) (GenBank® Accession No. AY525610) (25, 26), RASSF1A (nt −73 to +97) (GenBank® Accession No. DQ444319) (27), HIN-1 (nt −172 to nt −37) (GenBank® Accession No. AY040564) (28), and CRBP1 (nt −45 to nt +65) (GenBank® Accession No. X07437) (29). Nucleotide positions are relative to the transcriptional start site for each gene. MS-PCR primers and conditions are listed in Table 1.

### Example 3

### Statistical Methods

The Wilcoxon Rank-Sums test was used to compare the mean ranks of each covariate [median age, body mass index (BMI), Gail Score, probability of BRCA1/2 mutation and Masood score] according to positive or negative marker methylation status. Independently, the proportion of pre-menopausal and Caucasian women with a methylated marker was compared using the Pearson Chi-Square test.

Hierarchical clustering of gene methylation patterns was performed in the R statistical environment using complete linkage of correlations for symmetric binary data (30). Pair-wise correlations in gene methylation were examined using Fisher's Exact Test. To correct for the 45 multiple comparisons, p-values are adjusted using the Benjamini-step-up method for controlling the False Discovery Rate (FDR) (10).

The Wilcoxon Rank-Sums test was used to compare the mean ranks of the number of methylation events, age, and BRCAPRO scores in subjects testing negative as opposed to testing positive for BRCA1/2 mutation. The correlation between the number of promoter methylation events and BMI, age, Gail Model Score, and probability of an individual having a BRCA1/2 mutation (BRCAPRO score) was tested using the Spearman Rank Correlation coefficients. The Wilcoxon Rank-Sums test was used to test for differences in the mean ranks of the number of positive markers in Caucasians compared to African-Americans as well as pre-menopausal compared to peri-/post-menopausal women.

### Example 4

### Study Demographics

Study subject demographics are listed in Table 2. The initial RPFNA sample was tested from 109 women who 1) underwent RPFNA at Duke University Medical Center from Mar. 1, 2003 to Oct. 1, 2007 and 2) had sufficient epithelial cells for cytological testing. Seventy-seven percent (84/109) of subjects had bilateral RPFNA. Unilateral RFPNA was performed on women with prior mastectomy and RPFNA was not performed on irradiated breast tissue; therefore, 23% of subjects (25/109) had unilateral RPFNA. Ninety percent (98/109) of the women were Caucasian and 10% (11/109) were African-American. Twenty-nine unaffected pre-menopausal women with a familial pattern of breast cancer underwent BRCA1/2 mutation testing; thirty-four percent (10/29) of women tested positive for either a BRCA1 (8/10) or BRCA2 (2/10) mutation.

### Example 5

### Methylation Analysis of RPFNA Cytology

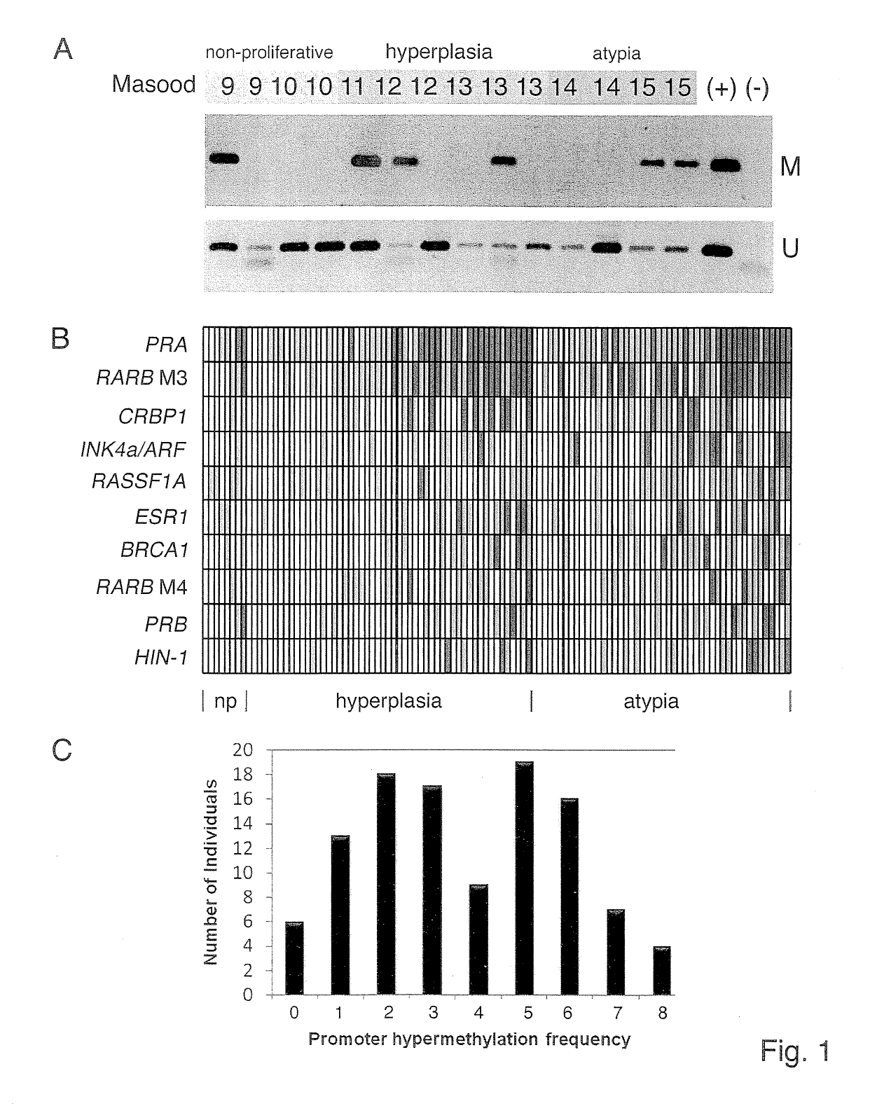

Promoter methylation of ten breast cancer-associated genes was evaluated in the initial RPFNA cytology from 109 high-risk women; 193 RPFNA samples were tested. Promoter targets included RARB (M3 and M4 sites), ESR1, INK4a/ARF, BRCA1, PRA, PRB, RASSF1A, HIN-1, and CRBP1. To perform this analysis, subjects were considered methylated for an individual marker if promoter methylation was detected in RPFNA cytology from either 1) one breast (unilateral methylation) or 2) both breasts (bilateral methylation). Representative RPFNA promoter methylation testing is presented in FIG. 1A. The distribution of each marker is presented in Table 3 and FIG. 1B. The median number of positive promoter methylation events per individual was 4 and the mean number was 3.75. Among the ten genes tested, the most frequently methylated genes in RPFNA cytology from high-risk women were PRA (125/190; 65.8%) and RARB M3 (112/193; 58.0%). The least frequently methylated genes were HIN-1 (29/190; 15.3%) and PRB (30/190; 15.8%).

### Example 6

### Bimodal Distribution of Promoter Methylation Provides Evidence for CIMP

The current definition of CIMP is based on the specific methylation of multiple CpG islands and implies a distinction between individuals with or without an enhanced pathological rate of somatic DNA promoter methylation. The distribution of promoter methylation events is shown in FIG. 1C. A bimodal distribution of methylation events in RPFNA cytology from high-risk women was observed.

### Example 7

### Specific Promoter Methylation Events are Associated with Abnormal Masood Score but not Age

Two types of methylation patterns have been previously described in colorectal cancer: aging-specific methylation and cancer-specific methylation (9). The present studies tested for both age-related methylation and whether specific promoter methylation events predicted abnormal RPFNA cytology. Promoter methylation in RFPNA cytology from 109 subjects was compared with age and Masood Cytology Index score (Table 4). While overall methylation events increased with age (p<0.0001), no specific methylation marker was associated with increasing age. In contrast, methylation of RARB M4 (p=0.051), INK4a/ARF (p=0.042), HIN-1 (p=0.044), and PRA (p=0.032) as well as the overall number of methylation events (p=0.004) were associated with increased Masood Cytology Index score. These data show that while the overall frequency of methylation events increases with age, methylation of the regions identified herein of the specific genes, RARB M4, INK4a/ARF, HIN-1, and PRA, is associated with an abnormal Masood Cytology Index score in mammary epithelial cells from high-risk women. These data provide evidence that specific methylation events are associated with early mammary carcinogenesis.

### Example 8

### Hierarchical Cluster of RPFNA Promoter Methylation

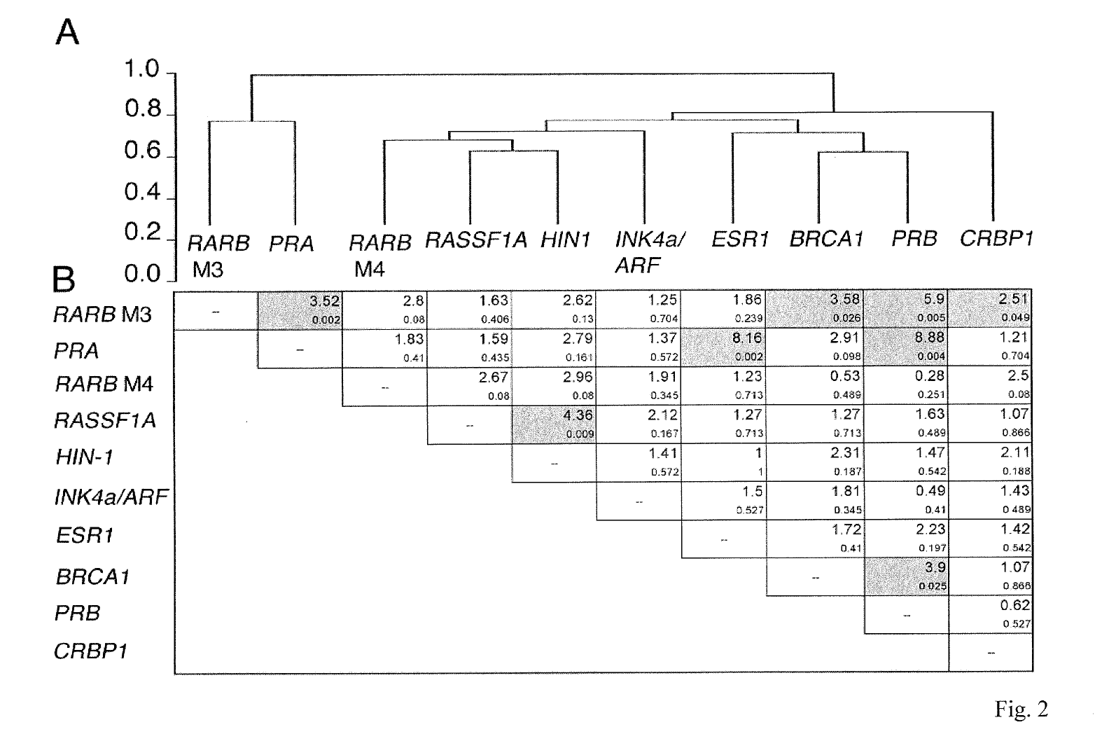

The clustering of methylation patterns in the ten genes was generated from all bilateral observations that did not have any missing values (n=189). A symmetric binary distance was used as the measure of pair-wise correlation and complete linkage was used to build the agglomerative tree structure (FIG. 2A). A hierarchical clustering of RARB M4, HIN-1, PRB, and INK4a/ARF was observed. As described above, methylation of RARB M4, HIN-1, PRB, and INK4a/ARF at the regions described herein was also associated with increased Masood Cytology Index scoring (Table 4).

### Example 9

### Associations Between Methylation Markers

The association between all pair-wise combinations of methylation markers was examined in the 189 samples through Fisher's Exact test for the odds ratio. FIG. 2B provides the estimated odds ratio and adjusted p-values for all 45 comparisons. After correcting for the FDR at the alpha=0.05 level, specific associations were observed between promoter methylation of 1) RARB M3 and BRCA1, PRB, and CRBP1 and 2) RASSF1A and HIN-1.

### Example 10

### Associations Between Promoter Methylation and Clinical Parameters

The association between promoter methylation and menopausal status, race, Gail model risk score, and BRCAPRO model score (Table 4) was tested. Due to the limitations of the Gail model, only 61% (67/109) of subjects could be assessed. A Gail Model score was not calculated in 42 individuals because of a prior history of contralateral breast cancer, ductal carcinoma in situ, because the subject was <35 years of age, or due to the Gail model underestimating risk in African-American subjects. The median number of positive methylation events in Caucasian versus African-American women was 3.5 versus 4.0, respectively (p=0.68). The number of methylation events in pre-menopausal women versus peri-/post-menopausal women was 3.0 versus 4.0, respectively (p=0.12). There was no statistically significant association found between the overall number of promoter methylation events and BMI (r=−0.14; p=0.32), Gail model risk score (r=−0.024; p=0.85), or BRACAPRO model score (BRCAPRO1: r=−0.074, p=0.41; BRCAPRO2: r=0.003, p=0.97). However, associations were observed between specific promoter methylation events and clinical variables. BMI was significantly lower in women displaying methylation of CRBP1 promoter (p=0.005) and a higher proportion of pre-menopausal women exhibited promoter methylation of PRB (p=0.030). There was a significant association between lack of promoter methylation of INK4a/ARF and both BRCAPRO1 and BRCAPRO2 scores (p=0.010 and 0.031, respectively).

The number of African American (i.e., black; Negro) women is relatively small but the representation is proportional to the catchment area. The incidence and distribution of methylation is equal in Caucasian and African American women.

### Example 11

### High Frequency of Promoter Methylation is Observed in Unaffected Women Who Tested Negative for a BRCA1/2 Mutation

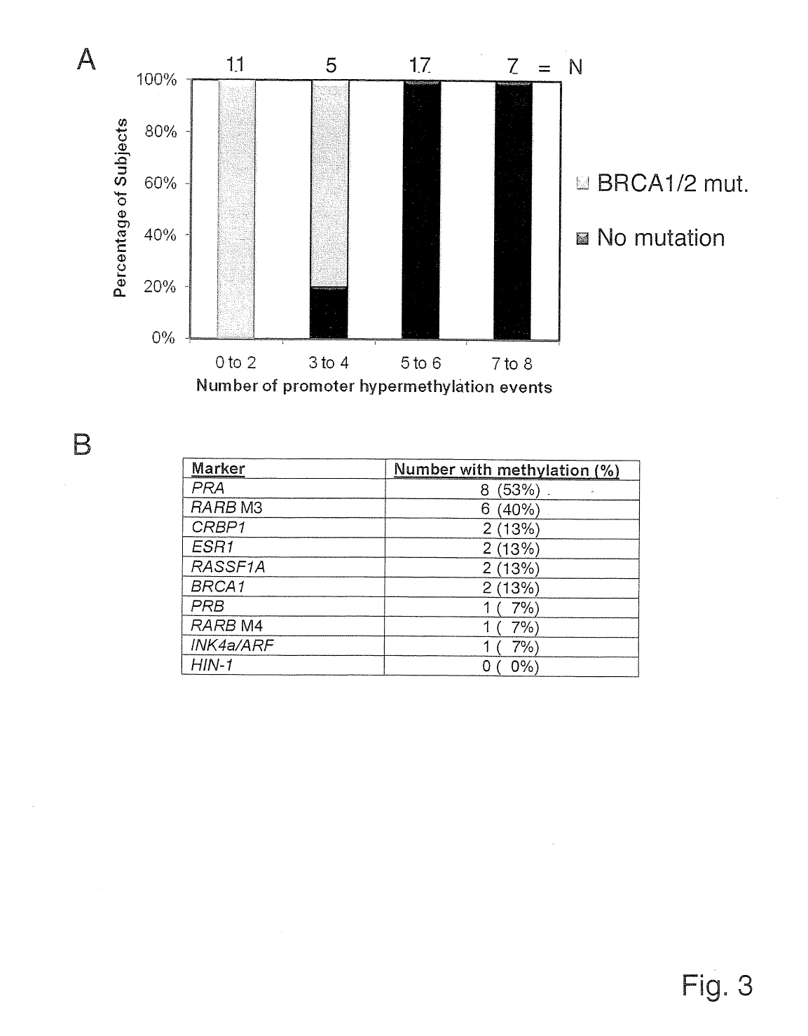

Women with a familial pattern of breast cancer were tested to observe whether there was an association between promoter methylation frequency and the presence or absence of a BRCA1/2 mutation. Forty unaffected women in the cohort underwent BRCA1/2 mutation testing. To be eligible for BRCA1 mutation testing, women were required to have a pre-test probability of ≧0.05. The 40 high-risk women tested 1) were pre-menopausal, 2) had significant family history of breast cancer, and 3) were unaffected. Twenty-five women (25/40) tested negative for both BRCA1 and BRCA2 mutations and fifteen (15/40) women tested positive for either a BRCA1 or BRCA2 mutation. The distribution of methylation events for women with or without BRCA1/2 mutation is shown in FIG. 3A, and the number and percentage of women methylated for each marker out of the 15 women testing positive for BRCA1/2 mutations is shown in FIG. 3B. In the group of women testing positive for BRCA1/2, 15/15 women had ≦4 methylated promoter/promoter element events, whereas only one (1/25) woman testing negative for BRCA1/2 had ≦4 methylated promoter/promoter element events (p<0.0001).

No woman with a BRCA1/2 mutation demonstrated methylation of HIN-1 and of women with a BRCA1/2 mutation, only one woman each demonstrated methylation of the RARB M4, INK4a/ARF, or PRB genes at the regions described herein. In contrast, eight women with a BRCA1/2 mutation exhibited methylation of PRA and six women with a BRCA1/2 mutation exhibited methylation of RARB M3. The median age of women testing positive for BRCA1/2 is 41, while the median age for those testing negative is 44 (p=0.43). These observations provide evidence that in unaffected high-risk women with a familial pattern of inherited breast cancer, there is an increased frequency of methylation events in women who test negative for a BRCA1/2 mutation and a decreased frequency of methylation events in women with a BRCA1/2 mutation. These data provide the first evidence for CIMP in non-BRCA-associated mammary carcinogenesis.

The foregoing is illustrative of the present invention, and is not to be construed as limiting thereof. The invention is defined by the following claims, with equivalents of the claims to be included therein. All publications, patent applications, patents, patent publications, sequences identified by Genbank® database accession numbers and other references cited herein are incorporated by reference in their entireties for the teachings relevant to the sentence and/or paragraph in which the reference is presented.

### REFERENCES

1. Issa, J P. “CpG island methylator phenotype in cancer.” Nat Rev Cancer 2004; 4:988-993.
2. Kondo, Y and Issa, J P. “Epigenetic changes in colorectal cancer.” Cancer Metastasis Rev 2004; 23:29-39.
3. Toyota, M, Ahuja, N, Ohe-Toyota, M, Herman, J G, Baylin, S B, and Issa, J P. “CpG island methylator phenotype in colorectal cancer.” Proc Natl Acad Sci USA 1999; 96:8681-8686.
4. Curtin, K, Slattery, M L, Ulrich, C M, et al. “Genetic polymorphisms in one-carbon metabolism: associations with CpG island methylator phenotype (CIMP) in colon cancer and the modifying effects of diet.” Carcinogenesis 2007; 28:1672-1679.
5. Kawasaki, T, Nosho, K, Ohnishi, M, et al. “Correlation of beta-catenin localization with cyclooxygenase-2 expression and CpG island methylator phenotype (CIMP) in colorectal cancer.” Neoplasia 2007; 9:569-577.
6. Ogino, S, Kawasaki, T, Kirkner, G J, Kraft, P, Loda, M, and Fuchs, C S. “Evaluation of markers for CpG island methylator phenotype (CIMP) in colorectal cancer by a large population-based sample.’ J Mol Diagn 2007; 9:305-314.
7. Ogino, S, Kawasaki, T, Ogawa, A, Kirkner, G J, Loda, M, and Fuchs, CS. “TGFBR2 mutation is correlated with CpG island methylator phenotype in microsatellite instability-high colorectal cancer.” Hum Pathol 2007; 38:614-620.
8. Weisenberger, D J, Siegmund, K D, Campan, M, et al. “CpG island methylation phenotype underlies sporadic microsattelite instability and is tightly associated with BRAF mutation in colorectal cancer.” Nat Genet 2006; 38:787-793.
9. Toyota, M and Issa, J P. “CpG island methylator phenotypes in aging and cancer.” Semin Cancer Biol 1999; 9:349-357.
10. Bae, Y K, Brown, A, Garrett, E, et al. “Methylation in histologically distinct classes of breast cancer.” Clin Cancer Res 2004; 10:5998-6005.
11. Bean, G R, Kimler, B F, and Seewaldt, V L. “Long-term raloxifene in a woman at high risk for breast cancer.” N Engl J Med 2006; 355:1620-1622.
12. Bean, G R, Scott, V, Yee, L, et al. “Retinoic acid receptor-beta2 promoter methylation in random periareolar fine needle aspiration.” Cancer Epidemiol Biomarkers Prey 2005; 14:790-798.
13. Fabian, C J, Kimler, B F, Zalles, C M, et al. “Short-term breast cancer prediction by random periareolar fine-needle aspiration cytology and the Gail risk model.” J Natl Cancer Inst 2000; 92:1217-1227.
14. U.T. Southwestern Medical Center at Dallas Presents CancerGene with BRCAPRO [homepage on the Internet]. Dallas (TX): The University of Texas Southwestern Medical Center at Dallas. c2004 [updated 2007 Jul. 30; cited 2008 Feb. 20]. Available from: http://www4.utsouthwestern.edu/breasthealth/cagene/.
15. Breast Cancer Risk Assessment Tool [homepage on the Internet]. Bethesda (MD): National Cancer Institute; c2000 [cited 2006 Jan. 13]. Available from: http://www.cancer.gov/bcrisktool/.
16. Euhus, D M. “Understanding mathematical models for breast cancer risk assessment and counseling.” Breast J2001; 7:224-232.
17. Parmigiani, G, Berry, D, and Aguilar, 0. “Determining carrier probabilities for breast cancer-susceptibility genes BRCA1 and BRCA2.” Am J Hum Genet 1998; 62:145-158.
18. Zalles, C M, Kimler, B F, Kamel, S, McKittrick, R, and Fabian, C J. “Cytology patterns in random aspirates from women at high and low risk for breast cancer.” Breast J 1995; 1:343-349.
19. Masood, S, Frykberg, E R, McLellan, G L, Scalapino, M C, Mitchum, D G, and Bullard, J B. “Prospective evaluation of radiologically directed fine-needle aspiration biopsy of nonpalpable breast lesions.” Cancer 1990; 66:1480-1487.
20. Seewaldt, V L, Johnson, B S, Parker, M B, Collins, S J, and Swisshelm, K. “Expression of retinoic acid receptor beta mediates retinoic acid-induced growth arrest and apoptosis in breast cancer cells.” Cell Growth Differ 1995; 6:1077-1088.
21. Baldwin, R L, Nemeth, E, Tran, H, et al. “BRCA1 promoter region methylation in ovarian carcinoma: a population-based study.” Cancer Res 2000; 60:5329-5333.
22. Lapidus, R G, Nass, S J, Butash, K A, et al. “Mapping of ER gene CpG island methylation-specific polymerase chain reaction.” Cancer Res 1998; 58:2515-2519.
23. Bean, G R, Bryson, A D, Pilie, P G, et al. “Morphologically normal-appearing mammary epithelial cells obtained from high-risk women exhibit methylation silencing of INK4a/ARF.” Clin Cancer Res 2007; 13:6834-6841.
24. Herman, J G, Graff, J R, Myohanen, S, Nelkin, B D, and Baylin, S B. “Methylation-specific PCR: a novel PCR assay for methylation status of CpG islands.” Proc Natl Acad Sci USA 1996; 93:9821-9826.
25. Kastner, P, Krust, A, Turcotte, B, et al. “Two distinct estrogen-regulated promoters generate transcripts encoding the two functionally different human progesterone receptor forms A and B.” Embo J 1990; 9:1603-1614.
26. Sasaki, M, Dharia, A, Oh, B R, Tanaka, Y, Fujimoto, S, and Dahiya, R. “Progesterone receptor B gene inactivation and CpG methylation in human uterine endometrial cancer.” Cancer Res 2001; 61:97-102.
27. Burbee, DG, Forgacs, E, Zochbauer-Muller, S, et al. “Epigenetic inactivation of RASSF1A in lung and breast cancers and malignant phenotype suppression.” J Natl Cancer Inst 2001; 93:691-699.
28. Krop, I E, Sgroi, D, Porter, D A, et al. “HIN-1, a putative cytokine highly expressed in normal but not cancerous mammary epithelial cells.” Proc Natl Acad Sci USA 2001; 98:9796-9801.
29. Shutoh, M, Oue, N, Aung, P P, et al. “DNA methylation of genes linked with retinoid signaling in gastric carcinoma: expression of the retinoid acid receptor beta, cellular retinol-binding protein 1, and tazarotene-induced gene 1 genes is associated with DNA methylation.” Cancer 2005; 104:1609-1619.
30. Krop, I, Maguire, P, Lahti-Domenici, J, et al. “Lack of HIN-1 methylation in BRCA1-linked and “BRCA1-like” breast tumors.” Cancer Res 2003; 63:2024-2027.

TABLE 1
MS-PCR primers and conditions

| | | | | | |
|---|---|---|---|---|---|
| | | | | Annealing | |
| Gene | Orientation | Primer Sequence | bp | Temp | SEQ ID NO |

| | | | | | |
|---|---|---|---|---|---|
| RARB M3 | Forward | 5′-GGT TAG TAG TTC GGG TAG GGT TTA TC | 234 | 57° C. | SEQ ID NO: 1 |
| | Reverse | 5′-CCG AAT CCT ACC CCG ACG | | | SEQ ID NO: 2 |
| RARB U3 | Forward | 5′-TTA GTA GTT TGG GTA GGG TTT ATT | 232 | 57° C. | SEQ ID NO: 3 |
| | Reverse | 5′-CCA AAT CCT ACC CCA ACA | | | SEQ ID NO: 4 |
| RARB M4 | Forward | 5′-GTC GAG AAC GCG AGC GAT TC | 148 | 56° C. | SEQ ID NO: 5 |
| | Reverse | 5′-CGA CCA ATC CAA CCG AAA CG | | | SEQ ID NO: 6 |
| RARBU4 | Forward | 5′-GAT GTT GAG AAT GTG AGT GAT TT | 150 | 57° C. | SEQ ID NO: 7 |
| | Reverse | 5′-AAC CAA TCC AAC CAA AAC A | | | SEQ ID NO: 8 |
| ESR1 M | Forward | 5′-GTG TAT TTG GAT AGT AGT AAG TTC GTC | 118 | 56° C. | SEQ ID NO: 9 |
| | Reverse | 5′-CGT AAA AAA AAC CGA TGT AAC CG | | | SEQ ID NO: 10 |
| ESR1 U | Forward | 5′-GGT GTA TTT GGA TAG TAG TAA GTT TGT | 120 | 52° C. | SEQ ID NO: 11 |
| | Reverse | 5′-CCA TAA AAA AAA CCA ATC TAA CCA | | | SEQ ID NO: 12 |
| INK4a/ARF M | Forward | 5′-TTA TTA GAG GGT GGG GCG GAT CGC | 150 | 63° C. | SEQ ID NO: 13 |
| | Reverse | 5′-GAC CCC GAA CCG CGA CCG TAA | | | SEQ ID NO: 14 |
| INK4a/ARF U | Forward | 5′-TTA TTA GAG GGT GGG GTG GAT TGT | 151 | 57° C. | SEQ ID NO: 15 |
| | Reverse | 5′-CAA CCC CAA ACC ACA ACC ATA A | | | SEQ ID NO: 16 |
| BRCA1 M | Forward | 5′-GGT TAA TTT AGA GTT TCG AGA GAC G | 182 | 63° C. | SEQ ID NO: 17 |
| | Reverse | 5′-TCA ACG AAC TCA CGC CGC GCA ATC | | | SEQ ID NO: 18 |
| BRCAI U | Forward | 5′-GGT TAA TTT AGA GTT TTG AGA GAT G | 182 | 63° C. | SEQ ID NO: 19 |
| | Reverse | 5′-TCA ACA AAC TCA CAC CAC ACA ATC A | | | SEQ ID NO: 20 |
| PRA M | Forward | 5′-ACG GGT TAT TTT TTT TTC G | 99 | 52° C. | SEQ ID NO: 21 |
| | Reverse | 5′-TAA AAT ATA CGC CCT CCA CG | | | SEQ ID NO: 22 |
| PRA U | Forward | 5′-ATG GGT TAT TTT TTT TTT G | 99 | 50° C. | SEQ ID NO: 23 |
| | Reverse | 5′-TAA AAT ATA CAC CCT CCA CA | | | SEQ ID NO: 24 |
| PRB M | Forward | 5′-TGA TTG TCG TTG GTA GTA CG | 200 | 59° C. | SEQ ID NO: 25 |
| | Reverse | 5′-CGA CAA TTT AAT AAC ACG CG | | | SEQ ID NO: 26 |
| PRB U | Forward | 5′-TGA TTG TTG TTT GTA GTA TG | 200 | 53° C. | SEQ ID NO: 27 |
| | Reverse | 5′-CAA CAA TTT AAT AAC ACA CA | | | SEQ ID NO: 28 |
| RASSF1A M | Forward | 5′-GGG TTT TGC GAG AGC GCG | 167 | 60° C. | SEQ ID NO: 29 |
| | Reverse | 5′-GCT AAC AAA CGC GAA CCG | | | SEQ ID NO: 30 |
| RASSF1A U | Forward | 5′-GGT TTT GTG AGA GTG TGT TTA G | 167 | 58° C. | SEQ ID NO: 31 |
| | Reverse | 5′-CAC TAA CAA ACA CAA ACC AAA C | | | SEQ ID NO: 32 |
| HIN-1 M | Forward | 5′-GTT TAG TTT TGA GGG GGG GGC | 163 | 60.5° C. | SEQ ID NO: 33 |
| | Reverse | 5′-AAC TTC CTA CTA CGA CCG ACG | | | SEQ ID NO: 34 |
| HIN-1 U | Forward | 5′-ATT GTA AAG TGA AGG TGT GGG TT | 163 | 63° C. | SEQ ID NO: 35 |
| | Reverse | 5′-CCA ACT TCC TAC TAC AAC CAA CA | | | SEQ ID NO: 36 |
| CRBP-1 M | Forward | 5′-TTG GGA ATT TAG TTG TCG TCG TTT C | 110 | 57° C. | SEQ ID NO: 37 |
| | Reverse | 5′-AAA CAA CGA CTA CCG ATA CTA CGC G | | | SEQ ID NO: 38 |
| CRBP-1 U | Forward | 5′-GTG TTG GGA ATT TAG TTG TTG TTG TTT T | 110 | 58° C. | SEQ ID NO: 39 |
| | Reverse | 5′-ACT ACC AAA ACA ACA ACT ACC AAT ACT ACA | | | SEQ ID NO: 40 |

TABLE 2
Patient characteristics for RPFNA

| Women Enrolled in Study | | 109 |
|---|---|---|
| Bilateral RPFNA | | 84 |
| Unilateral RPFNA | | 25 |
| RPFNA Samples Collected | | 193 |
| Average Age & Range (years) | | 47 (29-65) |
| Race | Caucasian | 98 (90%) |
| | African-American | 11 (10%) |
| Menopausal Status | Post-menopausal | 42 (39%) |
| | Pre-/Peri-menopausal | 67 (61%) |

| | |
|---|---|
| Hormone Replacement Use | |
| Current | 0 (0%) |
| Ever-use | 13 (12%) |
| Never-use | 96 (88%) |
| Anti-Estrogen Therapy (at the time of RPFNA) | |
| Tamoxifen | 0 (0%) |
| Raloxifene | 0 (0%) |
| Aromatase Inhibitor | 0 (0%) |
| Family History of Breast Cancer | 52 (48%) |
| Prior Abnormal Biopsies | |
| ADH | 13 (12%) |
| LCIS | 4 (4%) |
| DCIS | 11 (10%) |
| Hx. Contralateral Br Ca | 16 (15%) |
| Tested for BRCA½ mutation | 40 (37%) |
| Tested positive | 15 (14%) |
| BRCA1 mutation | 13 (12%) |
| BRCA2 mutation | 2 (2%) |
| Tested negative | 25 (23%) |

RPFNA = Random Periareolar Fine Needle Aspiration;
ADH = atypical ductal hyperplasia;
LCIS = lobular carcinoma in situ;
DCIS = ductal carcinoma in situ;
Hx = history;
Br Ca = breast cancer.

TABLE 3
Frequency of RPFNA promoter
methylation in RPFNA cytology.

| Marker | | Frequency | Percent |
|---|---|---|---|
| RARB M3 | No | 81 | 41.97 |
| | Yes | 112 | 58.03 |
| | NA | 0 | |
| RARB M4 | No | 158 | 81.87 |
| | Yes | 35 | 18.13 |
| | NA | 0 | |
| ESR1 | No | 152 | 79.17 |
| | Yes | 40 | 20.83 |
| | NA | 1 | |
| INK4a/ARF | No | 150 | 77.72 |
| | Yes | 43 | 22.28 |
| | NA | 0 | |
| BRCA1 | No | 155 | 81.58 |
| | Yes | 35 | 18.42 |
| | NA | 3 | |
| PRA | No | 65 | 34.21 |
| | Yes | 125 | 65.79 |
| | NA | 3 | |
| PRB | No | 160 | 84.21 |
| | Yes | 30 | 15.79 |
| | NA | 3 | |
| RASSF1A | No | 148 | 77.89 |
| | Yes | 42 | 22.11 |
| | NA | 3 | |
| HIN-1 | No | 161 | 84.74 |
| | Yes | 29 | 15.26 |
| | NA | 3 | |
| CRBP1 | No | 137 | 72.11 |
| | Yes | 53 | 27.89 |
| | NA | 3 | |

RARB (M3 and M4), ESR1, INK4a/ARF, BRCA1, PRA, PRB, RASSF1A, HIN-1, and CRBP1.
RPFNA = Random Periareolar Fine Needle Aspiration;
No = lack of methylation;
Yes = presence of methylation;
NA = data not available.

That which is claimed is:

1. A method of identifying a female subject as having an increased risk of developing breast cancer, comprising detecting methylation of one or more CpG islands in the promoter of a HIN-1 gene, in the M4 region of the promoter element of a RARB gene and in the promoter of an INK4a/ARF gene in the subject, whereby the presence of methylation of one or more CpG islands in the promoter of the HIN-1 gene, in the M4 region of the promoter element of the RARB gene and in the promoter of the INK4a/ARF gene identifies the subject as having an increased risk of developing breast cancer.

2. The method of claim 1, further comprising detecting methylation of one or more CpG islands in the promoter of a PRA gene of the subject.

3. The method of claim 1, wherein the subject has been previously identified as being at high risk of developing breast cancer.

4. The method of claim 1, wherein the subject has not been previously identified as being at high risk of developing breast cancer.

5. The method of claim 1, wherein the subject is negative for a BRCA1 mutation.

6. The method of claim 1, wherein the subject is negative for a BRCA2 mutation.

7. The method of claim 1, wherein the subject is negative for both a BRCA1 mutation and a BRCA2 mutation.

8. The method of claim 1, wherein the subject is a human.

9. A kit for the detection of methylation of one or more CpG islands in the promoter of a HIN-1 gene, in the M4 region of the promoter element of a RARB gene and in the promoter of an INK4a/ARF gene, comprising reagents and primers for amplification of CpG-containing nucleic acid of the HIN-1 gene, the RARB M4 gene and the INK4a/ARF gene.

10. The kit of claim 9, further comprising reagents and primers for amplification of CpG-containing nucleic acid of the PRA gene.
